(19) United States
(12) Patent Application Publication
(10) Pub. No.: US 2009/0028895 A1
(43) Pub. Date: Jan. 29, 2009

(54) METHODS AND COMPOSITIONS FOR REDUCING FACIAL LINES AND WRINKLES

(76) Inventor: Walter P. Smith, Wellington, FL (US)

Correspondence Address: BROWN RUDNICK LLP, ONE FINANCIAL CENTER, BOSTON, MA, 02111, US

(21) Appl. No.: 11/947,465

(22) Filed: Nov. 29, 2007

Related U.S. Application Data
(60) Provisional application No. 60/962,496, filed on Jul. 27, 2007.

(51) Int. Cl. A61K 36/00; A61K 31/045; A61K 31/195; A61K 36/068; A61K 36/87; A61P 17/00; A61K 36/899; A61K 36/84; A61K 31/44; A61K 31/522

(52) U.S. Cl. 424/195.15; 424/725; 424/733; 424/750; 424/766; 424/773; 424/777; 514/263.23; 514/343; 514/561; 514/730

(57) ABSTRACT

A dietary supplement composition comprising a therapeutically effective amount of at least one sirt 1 activating agent, a therapeutically effective amount of at least one Qi activating agent, and a therapeutically effective amount of at least one adaptogen is disclosed. An optional herbal treatment for stress-related deep wrinkles comprises chronic oral administration for at least two weeks of a beverage containing an effective amount of a valerian root extract sufficient to provide a desired reduction in the appearance of the deep wrinkles. A photoprotective agent may also be included. Also included are a microcirculation increasing agent. Also optional are an herbal toner and topical cellulite product.

### RELATED APPLICATION INFORMATION

This patent application claims priority to U.S. Provisional Application No. 60/962,496, filed on Jul. 27, 2007.

### TECHNICAL FIELD

The present disclosure relates generally to the field of reducing the appearance of facial lines and wrinkles and more particularly to dietary supplement compositions and methods for reducing deep dynamic facial wrinkles, with and without related topical agents and supplemental associated ingestibles.

### BACKGROUND OF THE INVENTION

As humans age, metabolism slows down. The primary consequences of such aging include decreases in the ability of the epidermis to renew itself, as part of a general slowing of cell renewal rates. This slowing of cell renewal can result in a general thinning of the skin, both epidermis and dermis which can cause a loss of firmness, skin sagging, and more easily bruised skin. Similarly, wound healing times increase with age. Additionally, related conditions such as the accumulation of cellular damage including oxidative by-products, protein glycation and a disruption of energy metabolism and coordinated enzyme activities occurs.

Topical treatment with exfoliants can increase skin cell renewal rates and reverse some of the cosmetic changes associated with a slowing metabolism. Over time, exfoliants have been shown to thicken both the epidermis and dermis increasing skin firmness. The topical application of anti-oxidants has been shown to reduce skin surface peroxidation and other oxidative processes and reduce accumulated damage from sun exposure. Moreover, the topical application of vitamin B derivatives as well as vitamin C analogs and co-factors, has been an effective strategy to reduce age associated changes in wound healing.

### SUMMARY OF THE INVENTION

While topical treatments are an effective means to treat skin, nutritional supplements have also been suggested. U.S. Pat. No. 5,869,540 discloses the ingestion of valerian root and other muscle relaxing herbs for the purpose of directly influencing muscles in the skin to control deeper wrinkles. Additionally, published studies have suggested that lycopene and other anti-oxidants when ingested on a daily basis can modify skin properties, specifically the ability to resist UV damage. Oral Anti-Oxidant Complex Protects Against Ultraviolet Irradiation Induced Erythema and Immediate Pigment Darkening In Human Skin in Vivo. J Leverett et al J. Inv. Dermatol. vol. 127s, s56 2007. In addition a recent study has shown that the ingestion of very large quantities of squalene can increase skin moisture content. Effects of Dietary Squalene Supplementation on Skin Aging in Human Skin in Vivo. S. Cho et al. J. Inv. Dermatol. vol 127 supplement s39, 2007.

Historically, it has been shown that protein or essential fatty acid deficiencies can lead to structural changes to both skin and hair. These include a breakdown of skin barrier function leading to excess water loss and scaly skin, and dry brittle hair which can break easily. Supplementation of the diet with protein and essential fatty acids eliminates the deficiency and quickly restores the skin and hair back to normal conditions. Thus, a link between nutrition and skin conditions exist, but only when skin is abnormally deficient. More particularly, studies show skin problems, which are caused by lack of EFAs, are marked by a return to normal skin when a normal diet is restored. Ref. EFA deficiency in four adult patients, T J Richardson, Am. J. clin. Nutr 28: 258-293, 1975. It would be desirable to provide a dietary supplement composition that improves the skin's appearance with design parameters to increase cell renewal rates. It would also be desirable to provide a method and composition that helps ameliorate deep facial lines and wrinkles when topically applied and used in combination with the dietary supplement. It would also be desirable to provide a dietary targeting supplement that helps prevent further damage to skin which may eventually cause increased wrinkles.

A dietary supplement alone or in combination with topical compositions or supplemental ingestibles to help ameliorate fine lines and deep facial wrinkles is provided.

In accordance with the inventive method, a dietary supplement composition comprises a therapeutically effective amount of at least one sirt 1 activating agent, a therapeutically effective amount of at least one Qi activating agent, and a therapeutically effective amount of at least one adaptogen.

At least one sirt 1 activating agent may be selected from the group consisting of resveratrol, extracts containing resveratrol, such as grape skin, rhodiola rosea, Japanese Knotwood, fisetin, 4-isoleucine and wheat grass extract.

At least one Qi activating agent from the group consisting of schisandra berry, glycerrhiza uralenis or glabra, red peony, astragali root, angelica root and maca may be selected.

At least one adaptogen from the group consisting of holy basil, lepedium meyenil and withania somnifera may be selected.

The herbal treatment for stress-related deep wrinkles comprises chronic oral administration for at least two weeks of a beverage containing an effective amount of a valerian root extract sufficient to provide a desired reduction in the appearance of the deep wrinkles by a photoprotective agent and a photoprotective agent of lycopene.

Grape skin is believed to be effective in concentrations of 25 mg to 500 mg, with concentrations of 25 mg to 200 mg preferred. Rhodiola rosea is believed to be effective in concentrations of 25 mg to 200 mg, with concentrations of 25 mg to 500 mg preferred. Japanese Knotwood is believed to be effective in concentrations of 25 mg to 200 mg, with concentrations of 25 mg to 500 mg preferred. Sineftin is believed to be effective in concentrations of 25 mg to 200 mg, with concentrations of 25 mg to 500 mg preferred. Wheat grass extract is believed to be effective in concentrations of 10 mg to 500 mg, with concentrations of 10 mg to 200 mg preferred.

Schisandra berry is believed to be effective in concentrations of 10 mg to 500 mg, with concentrations of 25 mg to 100 mg preferred. Glycerrhiza uralenis is believed to be effective in concentrations of 10 mg to 500 mg, with concentrations of 25 mg to 100 mg preferred. Glabra is believed to be effective in concentrations of 10 mg to 500 mg, with concentrations of 25 mg to 100 mg preferred. Red Peony is believed to be effective in concentrations of 10 mg to 500 mg, with concentrations of 25 mg to 100 mg preferred. Astragali root is believed to be effective in concentrations of 25 mg to 100 mg, with concentrations of 10 mg to 500 mg preferred. Angelica root is believed to be effective in concentrations of 25 mg to 100 mg, with concentrations of 10 mg to 500 mg preferred. Maca is believed to be effective in concentrations of 25 mg to 100 mg, with concentrations of 10 mg to 500 mg preferred.

Holy basil is believed to be effective in concentrations of 25 mg to 250 mg, with concentrations of 25 mg to 500 mg preferred. Lepedium meyenil is believed to be effective in concentrations of 25 mg to 250 mg, with concentrations of 25 mg to 500 mg preferred. Withania somnifera is believed to be effective in concentrations of 25 mg to 500 mg, with concentrations of 25 mg to 250 mg preferred.

Adenosine is believed to be effective in concentrations of 1 mg to 50 mg, with concentrations of 2 mg to 10 mg preferred. Cordyceps sinensis is believed to be effective in concentrations of 1 mg to 50 mg, with concentrations of 2 mg to 10 mg preferred. Nicotine is believed to be effective in concentrations of 0.2 mg to 5 mg. Caffeine is believed to be effective in concentrations of 0.1 mg to 25 mg, with concentrations of 5 mg to 15 mg preferred.

Resveratrol is believed to be effective in concentrations of 10 mg to 200 mg, with concentrations of 10 mg to 500 mg preferred.

If one of the above materials will perform two functions, its total concentration is in the range specified above, for example, 50 mg of wheat grass will be substantially effective in functioning as a sirt 1 activating agent and as a Qi activating agent. Moreover, some of the above materials must be administered in very large quantities before toxicity issues are raised and this represents the true upper limit of the dose.

### DETAILED DESCRIPTION OF EMBODIMENTS OF THE INVENTION

It has been tested and observed that oral ingestion of a select group of botanical actives can increase skin cell renewal rates and by this action reduce lines and wrinkles on mature test subjects. According to the present disclosure, it may be desirable to include at least one material from each of the classes of active materials described below. It has also been learned in accordance with the invention that topical application with or without supplemental ingestibles is effective in addressing wrinkles and the appearance of aging.

### Ingestible Composition

The first class of materials includes those with direct elevating effects on the sirt 1 protein. The sirt 1 protein is important in mitochondria metabolism which generates the energy for facial cell functions. During aging, energy production in cells decreases due to an under expression of the sirt 1 protein. Some sirt 1 activating materials include resveratrol and extracts containing resveratrol such as grape skin, Japanese Knotwood and wheat grass extract. It has also been observed in accordance with the invention that Rhodiola rosea, although not containing significant amounts of resveratrol falls into this category of actives. Similarly, fisetin or 4-isoeucine may act as a sirt 1 activator similar to resveratrol.

According to the present disclosure, rhodiola rosea with a minimum composition of 3% rosavins and 0.8-1% salidroside is used. This dry lyophilized powder composition may be provided by, for example, Natural and Marine, Inc.™, of Paramus, N.J., USA, under the name Rhodiola extract. Siberian rhodiola rosea extract has been shown to reduce and/or prevent stress-induced cardiac damage, decrease myocardial catecholamines and cyclic adenosine monophosphate (cAMP) levels. It also reduces adrenal catecholamine release, activates μ-opiate receptors in heart muscle and thus, helps prevent reperfusion arrhythmias. Similarly, a hydroalcoholic grape extract from grape (red) skin with a composition of 10% minimum resveratrol as provided under the product name grape extract by Active Organics™ of Lewisville, Tex. may be used.

Fisetin is a flavonoid derived from strawberries and also found in tomatoes. It is about equal in activity to resveratrol regarding sirt 1 stimulation. Fisetin can be used in place of resveratrol at 5-25 mg, or combined with resveratrol. If a 20% fisetin strawberry extract is used, it should be used at 100 mg. A suitable strawberry extract is available from Tryphyto Industries, Port Washington, N.Y.

Another sirt 1 activator is 4-isoleucine and is available from chemical suppliers such as Sigma or Aldrich. 4-isoleucine can be used in a similar fashion as resveratrol or fisetin. It can be used at 5 mg per day alone or in combination with one or more of the sirt 1 activators.

The second class of active materials include Qi activating herbs such as schisandra berry, glycerrhiza uralenis or glabra, red peony, astragali root, angelica root and maca. These materials have been reported to increase mitochondrial enzymes involved in energy production or increase ATP synthesis. In the cellular mitochondria, energy is produced for all cellular functions. Energy (ATP) is mainly produced aerobically via the Krebs cycle and anaerobically via glycolysis. In the skin most energy is produced via glycolysis since oxygen levels are quite low, especially in the lower epidermal and dermal layers where energy production takes place. The Krebs cycle relies on a number of key enzymes to produce and store ATP. Glycolysis also is dependent on a series of enzymes involved in energy production. The enzyme 1,6 bis-fructose phosphatase is generally thought to be the critical and rate limiting enzyme in the production of ATP through glycolysis. In theory, ingestion of the Qi activating herbs enhances mitochondrial energy production through the activation of this critical enzyme. Qi activating materials are essential to traditional Chinese medicine and have been implicated in a number of health processes, however, their ability to improve cell renewal rates is generally unexplored.

For example, schisandra berry (ocimum sanctum, schisandra chinensis) having a composition with a 1% schisandrol B minimum, such as that provided by Natural and Marine Resources as dry lyophilized powders may be used in accordance with the present invention. Also, maca root (lepidium meeyenii) with a composition of >1% glycoalkaloids as provided by Active Organics (as a hydroalcoholic concentrated liquid extract) may be used in accordance with the present disclosure. Similarly, licorice root (glycyrriza glabra) having a composition of >5% glycyrrhizin such as that provided by Centerchem, Inc.™ may be used in accordance with the present disclosure. Likewise, red peony root (paeonia lactiflora) having a composition of >1% paeoniflorin such as that proved by Natural and Marine Resources may be used. Also, astragalus root (astragalus membranaceus) at >1% alkaloids as provided by Natural and Marine Resources as a dry powder may be used. Angelica root (angelica pubescens) have >1% tannins and >0.1% angelic acid as provided by Natural and Marine Resources as a dry powder have exhibited activities that may reduce pain, dilate blood vessels and relax uterine muscles.

Additionally, a third class of active materials used in accordance with the present disclosure includes the adaptogens. This class includes holy basil (ocicmun sanctum), lepedium meyenil and withania somnifera. Adaptogens are balancing bio-molecules. These materials, usually herbs, have the potential of increasing cellular activities that are over expressed and increasing cellular functions which are diminished. Similarly, these materials have been implicated in the treatment of a variety of human disease processes, however, they have not been applied in the area of cell renewal rates and skin. For example, holy basil leaf (ocimum sanctum) with a composition of 2.5% ursolic acid minimum as is provided by Natural and Marine Resources as a lyopholized powder may be used in accordance with the present disclosure. Holy basil, also known as tulsi, is generally used in ayurvedic remedies for common colds, headaches, stomach disorders, inflammation, heart disease, various forms of poisoning and malaria. Another adaptogen that may be used in accordance with the present disclosure is ashwagandha root (withania somnifera) of a composition of >5% wiffianolides such as that available from Natural and Marine Resources as a lyopholized dry powder.

A forth class of active materials used according to the present disclosure are those designed to increase microcirculation into the skin such as adenosine, cordyceps sinensis, nicotine, caffeine or any ingestible material capable of elevating skin blood flow at 30-50% for several hours as determined by a Doppler laser blood flow measurement. Blood flow to the skin decreases with age usually due to photo damage to the vascular system supporting microcirculation into the dermis. One such microcirculation increasing material that may be used is cordyceps mycelia (cordyceps sinensis) having a composition of >0.15% adenosine such as that provided by Active Organics™.

The above materials may also be supplemented with a fifth class of active materials, namely antioxidants. A blend of anti-oxidants such as lycopene and/or Lyco-mato™ available from Lycored Corp., Organge N.Y. 07040 isolated from tomatoes, or polyphenols, isolated from green tea or white tea, are believed in accordance with the invention to enhance the inventive wrinkle reducing effect by preventing inflammation and reducing future damage.

### Cell Renewal Measurement

Several test methods were used to evaluate results of the compositions according to the present disclosure. One test method used was the skin cell renewal (dansyl chloride) method. The rate of cell renewal, or the transit time for an epidermal basal cell, to mature and slough off the skin surface can be assessed via a dansyl chloride staining method. As we age the rate of renewal decreases from about 15-20 days for a thirty year-old to about 30 days for a 70 year-old.

To assess skin cell renewal rates, between eight (8) and twelve (12) subjects were recruited and had refrained from using any moisturizer product and cleansing products on the volar forearm for 5 days prior to and throughout the course of the study. Subjects had no adverse skin conditions and could be no greater than 65 years of age.

The full thickness epidermis was stained with a fluorescent dye as follows: 5% ultra pure dansyl chloride was milled into petrolatum and applied as a very thin film to standard adhesive bandages that were 2 cm by 3 cm. Six to eight test sites, per subject (plus two control areas as detailed below), 3-4 per arm, were marked on the volar forearm and the band aids were firmly and snugly applied to each test site. The band aids remained undisturbed, without washing, on each subject for 24 hours. After 24 hours, the band aids were removed and the sites were washed to remove residual stain. Staining was confirmed by viewing the sites with a long wave (ultra violet) UV light source to detect dansyl chloride fluorescence. In accordance with the dansyl chloride test, cell renewal time is taken as the time it takes for the stained epidermis to be shed i.e., the time for the stain to disappear.

To assess product effects on cell renewal rates, test products were applied topically twice a day in a randomized fashion to the test sites with an untreated site serving as a control. Application was typically twice-a-day unless otherwise indicated. Removal of the stain, reflecting loss of stratum corneum was monitored daily with the UV light source until complete removal, usually occurring in 3-4 weeks, of all stain was noted. The percentage increase in cell renewal was calculated as follows:

$$\% Change in Cell renewal = \frac{[ \begin{matrix}( days for cell renewal ( untreated ) - \\ ( days for cell renewal ( treated )\end{matrix} ]}{days for cell renewal ( untreated )} * 100$$

There were six to eight test sites on each subject, one or two sites served as untreated controls and one or two sites served as a standard positive control. A standard positive control of 5% lactic acid, pH 3.0 dissolved in water was an effective control yielding increases in renewal rates of about 25-30%.

### Global Aging Assessment.

Another test method used according to the present disclosure was the global age assessment. Skin age was assessed either full-face or half-face using a series of both visual and tactile facial cues, each of which was associated with aging. Some of the parameters used to assess age includes the following: lines and wrinkles (assessed via counting as below) skin texture (assessed by gently rubbing the finger on the skin surface), firmness (assessed probing and lift the cheek area with the finger), eye area puffiness (assessed by palpitating the under eye area), skin sagging and distention (lifting the cheek upwards) age spots and skin color homogeneity. After a careful analysis by clinicians, a global age was noted for each panelist. By “global age” is meant the age which the subject appears to be. For example, three fifty year old subjects may appear to be forty, fifty, and fifty-five, due to differences in skin type exposure to sun, and so forth. These subjects would have global ages of 40, 50 and 55 respectively. Rather than give a specific age, panelists were assigned to an age group (groups are spreads of five years). A point value was assigned to each group as follows:

| Age | | Points |
|---|---|---|
| <30 | years | 0 |
| 31-35 | | 1 |
| 36-40 | | 2 |
| 41-45 | | 3 |
| 46-50 | | 4 |
| 51-55 | | 5 |
| 56-60 | | 6 |
| 61-65 | | 7 |
| 66-70 | | 8 |
| >70 | | 9 |

During the tests according to the present disclosure, subjects were evaluated, and assigned a global age and thus a score (for example a global age of 53 would have a value of 5*). Scores were compared before and after cosmetic or nutritional treatments. A subject whose global age assessment went from 53 before, to 39 after treatment, would have numerical scores of 5* before treatment and 2* after treatment. This scale allows for quantification of changes in appearance (i.e. apparentage) before and after product treatments with a high degree of validity.

### Superficial Facial Lines

Fine, medium and deeper lines and wrinkles were assessed via the Packman method as described in; Paclanan, E., and Gans, E. H. Topical Moisturizers: Quantification of the Effect on Superficial Facial Lines. Soc. Cos. Chem. 29, 79-90 (1992). Essentially, wrinkles in the eye, mouth and forehead area were identified by number and depth and a total wrinkle score was computed. In a typical evaluation, the face was divided into five sections, the eye area, left and right, the forehead, and the mouth area, left and right. Wrinkles were graded in each area based upon number and depth. A numerical wrinkle score was obtained for each of the five facial areas based upon the following table and a total facial wrinkle score was obtained by the summing the numbers for the five facial sections.

### Quantification of Superficial Facial Lines (SFL)

| Number of wrinkles | Type of Wrinkle | Score |
|---|---|---|
| 0 | shallow, medium or deep | 0 |
| 1-3 | shallow | 1 |
| 1-3 | medium | 2 |
| 1-3 | deep | 3 |
| 4-6 | shallow | 2 |
| 4-6 | medium | 3 |
| 4-6 | deep | 4 |
| >7 | shallow | 3 |
| >7 | medium | 4 |
| >7 | deep | 5 |

### Panel Selection Criteria

According to the present disclosure subjects currently using or having used within the last six months retin-A, or analogs, corticosteroids, benzoyl peroxide, and topical anti-biotics were excluded. Subjects having had facial peels or dermabrasion within the last year were excluded. Other criteria for exclusion included subjects with psoriasis, eczema, or atopic dermatitis; subjects currently on test in any other clinical study for topical or systemic medications or products; subjects with known communicable disease; subjects who were pregnant or intend to become pregnant within the next 60 days; subjects who were nursing/lactating; and subjects who had botox, restyln or any other “rejuvenating” dermatologic treatment within the last year. Also excluded were subjects who may have expressed a negative reaction to a test sample based on medical or other pre-test condition. Subjects on medication were reviewed on a case-by-case basis.

As will be appreciated by those of skill in the art, the amount(s) of active ingredients which constitute a therapeutically effective dose will vary based on the depth, length and number of dynamic facial wrinkles, other skin and health conditions (past and present), and route(s) of administration. Dose frequency will also vary based on a number of factors such as the nature of the composition used, i.e., contains delivery enhancers, contains sustained release ingredients, age of the test subjects, season the test material is being used. Suitable dosing regimens can be readily selected by those skilled in the art, taking into consideration these factors. It is contemplated that such dosage variation shall follow the normal procedures and limits followed by health professionals in normal practice.

### Study 1: Positive Control(Ingestible Only)

Study 1 was used as a positive control and proof of concept validation to establish that nutritional supplements can influence skin properties. The ingestion of a skin energizing supplement combined with a photoprotective blend influenced both skin metabolism, as assessed by the dansyl chloride cell renewal assay, and increase skin photoprotection when measured by MED determination. See the protocol described in the Federal Register at Federal Register, Volume 64, No. 98 at page 27666 et seq. (May 1999).

Panel A consisted of an older group of test subjects with an average age of 59.8. Panel B consisted of a younger group with an average age of 19.3 years. Each of the members of Panel A and Panel B were directed to ingest a dose of the inventive energizing and protective supplement blend twice a day for ten weeks. Skin type was varied, however, the lightest and darkest skin types were avoided.

The oral dose of the inventive supplement included 100 mg rhodiola rosea, 25 mg grape extract with 10% resveratrol, 50 mg schisandra berry extract, and 75 mg lycopene. The composition was contained in a conventional two-piece hard shelled gelatin capsule.

Before and after the ten weeks skin cell renewal rates were determined by the dansyl chloride method. Results are shown in Table 1.

As can be seen in the following table, the inventive energizing blend resulted in a decrease in stratum corneum transit time, or increased cell renewal rates in older subjects. There was no effect on the younger subjects, probably because the turnover rate in young subjects is apparently at its maximum.

TABLE 1
Effect on Cell Renewal Rates with Nutritional Supplement
(days for Dansyl chloride stain to disappear)

| | Before | | | |
|---|---|---|---|---|
| Group | treatment | After 10 wks | Change | p Value |
| Panel A OLD | 24.8 days | 20.6 days | reduced 4.2 | <0.05 |
| Panel B YOUNG | 16.4 days | 16.0 days | reduced 0.4 | >0.5 |

Sun sensitivity in the studies described herein is a relative comparison of the time a defined UV exposure is necessary to create erythema or a sun burn. More time to create a sun burn means the skin is less sensitive to sun and would be more resistant to the aging effects of UV exposure. As. Table 2 shows both groups showed an increase in photoprotection due to the inclusion of lycopene, a known anti-oxidant.

Results from this study established proof of concept. A nutritional blend when used over an extended period of time can directly influence skin properties.

TABLE 2
Effect on Sun Sensitivity (Determination of MEDs)
(sun exposure time for erythema to develop)

| Group | Before treatment | After 10 wks | Change | p Value |
|---|---|---|---|---|
| Panel A | 45.8 sec | 51.2 sec | +5.4 | na |
| Panel B | 54.6 sec | 60.4 sec | +5.8 | |

### Study 2: Placebo Controlled 90 Day Nutritional Supplement to Assess Cell Renewal Rates and Lines and Wrinkles(Ingestible Only)

This study consisted of two groups of twenty subjects, each group having an average age of 50 years or above, with a minimum age of 45 years and a maximum of 60 years. Each group ingested a skin energizing nutritional supplement in accordance with the invention, or placebo, once a day for a three month (90 day) period. At the study start, after 45 days and after 90 days, subjects were evaluated for cell renewal rates, superficial lines and wrinkles, and relative age, as determined by the global age assessment method as described above.

The forty subjects were grouped into two panels. Panel A had an average global age assessment of 53 and Patel B had an average age of 53.35. All test subjects ingested a capsule of about 250 mg daily for ninety days. No particular skin type was chosen or omitted. The soft gelatin based capsule contained various material with respect to each panel. Panel A ingested 100 mg rhodiola rosea, 100 mg schisandra berry, 25 mg resveratrol, 25 mg holy basil, 5 mg adenosine according to our embodiment of the invention. Panel B ingested 200 mg of a 10% sucrose blended with inert carrier starch.

As the following Table 3, Table 4 and Table 5 demonstrate, while the placebo group showed no changes in skin cell renewal rates, the group receiving the blend according to the invention showed a significant decrease in transit time, in other words an increase in cell renewal rates. This increase was more than 11% after 90 days of treatment and was statistically significant (Table 3). No statistically significant changes were observed for the placebo group with respect to cell renewal rates. The group receiving the nutritional supplement also showed significant reductions in the appearance of superficial lines and wrinkles (Table 4), as well as showing a clinical reduction via global age assessment (Table 5). No such changes were observed in panel B the control group.

TABLE 3
Effect on Cell Renewal Rates with Nutritional Supplement Treatment
(days for dansyl chloride stain to disappear)

| Group | Before treatment | After 45 days | After 90 days |
|---|---|---|---|
| Panel A | 19.1 | 17.8** | 16.95** |
| Panel B (control) | 19.4 | 19.85 | 19.55 |

**Significant changes from baseline values
Scoring of superficial facial lines (“SFL”) were performed by the Inventor or clinicians trained in the method

TABLE 4
Effect on Superficial facial lines score with Nutritional
Supplement Treatment (Total SFL Score)

| Group | Before treatment | After 45 days | After 90 days |
|---|---|---|---|
| Panel A | 18.4 | no data | 16.5** |
| Panel B (control) | 18.7 | no data | 18.5 |

**Significant changes from baseline values

TABLE 5
Effect on Global Age Assessment with Nutritional Supplement
Treatment (Average Global Age Score)

| Group | Before treatment | After 45 days | After 90 days |
|---|---|---|---|
| Panel A | 5.2 | 4.85 | 4.5** |
| Panel B (control) | 5.15 | 4.95 | 5.05 |

**Significant changes from baseline values

### Study 3: Placebo Controlled 60 Day Nutritional Supplement Study and Topical Toner to Assess Cell Renewal Rates and Lines and Wrinkles

Eight groups of ten subjects, each group having an average age of greater than fifty years, ingested a skin energizing nutritional supplement(s) or placebo, once a day for a 60 day period. Certain subjects also used a prepared topical exfoliating toner as detailed below also designed to enhance skin cell renewal rates. At the study start and after 60 days, subjects, were evaluated for cell renewal times, superficial lines and wrinkles, and relative age, as determined by the above global age assessment method.

Eighty subjects were grouped into eight panels. Subjects ingested or were treated with the following compositions. Skin care products were applied once each day and oral supplements, a soft gelatin capsule containing the respective dose detailed below, were taken once each day in the morning.

TABLE 6

| Panel (Ave. Age) | Nutritional Product | Skin Care Product |
|---|---|---|
| A (55.1) | Placebo 10% Sucrose | none |
| B (53.5) | Placebo 10% Sucrose | Exfoliating toner |
| C (54) | Composition 1 | none |
| D (53.4) | Composition 2 | none |
| E (54.1) | Composition 3 | none |
| F (53.7) | Composition 3 | Exfoliating toner |
| G (54.8) | Composition 4 | none |
| H (53.9) | Composition 5 | none |

Subjects were evaluated before and after 60 days of treatment. Panel specific soft gelatin capsules were formulated with compositions as described below and taken once a day for 60 days.

Composition 1 consisted of 100 mg rhodiola, 200 mg maca, 50 mg holy basil, 5 mg adenosine. Composition 2 was 100 mg rholdiola, 50 mg schisandra berry, 50 mg lepedium, 50 mg cordyceps and 5 mg caffeine. Composition 3 was 50 mg resveratrol, 50 mg wheat grass, 50 mg red peony, 50 mg angelica, 50 mg withania, 5 mg adenosine. Composition 4 was 100 mg resveratrol, 100 mg schisandra berry, 50 mg withania. Composition 5 was 100 mg wheat grass, 25 mg red peony, 25 mg resveratrol, 25 mg withania, 25 mg maca and 25 mg cordyceps.

Table 7 shows the formulation for exfoliating toner used in Study 3.

TABLE 7

| | | |
|---|---|---|
| 1. | Deionized Water | 88.190% |
| 2. | Methyl Gluceth-20 | 1.000% |
| 3. | Potassium Sorbate | 0.100% |
| 4. | Sodium Benzoate | 0.100% |
| 5. | Phenoxyethanol | 0.600% |
| 6. | Citric acid | 0.010% |
| 7. | Mucor Miehei Extract, Butylene Glycol, | |
| | and N-Acetylglucosamine | 5.000% |
| | (Actizyme MCS, Active Organics)***** | |
| 8. | Grape skin extract | 1% |
| 9. | Schisandra | 1% |
| 10. | Holy basil | 1% |
| 11. | Adenosine | 0.1% |
| 12. | Withania | 1% |
| 13. | Maca | 0.9% |

To make the exfoliating toner, ingredients one through six are mixed in the sequence listed above at 80 degrees Celsius with agitation. When formula is smooth and homogenous, mixture is cooled to room temperature with agitation. When the formula reaches 40 degrees, mixing is continued until the batch is smooth and at room temperature.

Either 1% Lactic Acid, Glycolic Acid, Salicylic Acid, or combinations thereof can replace the mucor meihei extract as the exfoliant in the exfoliating toner as described in Table 7. The cell renewal exfoliating toner included a preparation of 1% grape skin extract (5% resveratrol), 1% schisandra, 1% holy basil, 0.1% adenosine, 1% withania and 0.9% maca.

The results of Study 3 are shown in Tables 8, 9 and 10. Study 3 shows that 60 days is sufficient to establish benefits. Additionally, study 3 shows that a variety of herbs with similar metabolic activities can be substituted for each other. Specifically Table 8 shows that several of the herbal compositions containing a variety of different herbs from the three active classes are similarly effective. This can be seen by comparing the data and compositions from Panels C, D, E, G and H. While five different compositions were used each had a stimulatory effect on cell renewal rates. Furthermore, there is a synergistic or at least cooperative interaction between the oral cell renewal supplement and the use of a topical cell renewal product such as an enzyme exfoliant or AHA material. This can be seen by comparing the results from Panels E and F. The data in Table 9 is consistent with that observed in Table 8, when a composition was able to increase cell renewal rates to a significant degree, we observed a significant reduction in the appearance of lines and wrinkles. Finally we also see in Table 10 that a clinical reduction in global age assessment was noted in each panel wherein a significant increase in cell renewal rates was observed. Thus, stimulating skin cell renewal rates via the inventive products results in the appearance of an overall improvement in the skin appearance related to age associated changes.

TABLE 8
Percent Change in Cell Renewal Rates with Oral and Topical Product
(days for Dansyl chloride stain to disappear)

| Group | Before treatment | After 60 days |
|---|---|---|
| Panel A (placebo) | 23.2 | 22.4 |
| Panel B (placebo) | 21.7 | 19.3** |
| Panel C | 22.3 | 18.7** |
| Panel D | 24 | 19.4** |
| Panel E | 23.1 | 17.9** |
| Panel F | 22 | 16.2** |
| Panel G | 22.5 | 17.5** |
| Panel H | 22.7 | 17.4** |

**Significant changes from baseline values

TABLE 9
Change in Superficial facial lines with Oral and Topical Product
(Total SFL Score)

| Group | Before treatment | After 60 days |
|---|---|---|
| Panel A (placebo) | 16.5 | 16.6 |
| Panel B (placebo) | 18 | 14.6** |
| Panel C | 17.7 | 16.1** |
| Panel D | 16.8 | 14.5** |
| Panel E | 18.8 | 17.1** |
| Panel F | 18.4 | 13.5** |
| Panel G | 17.6 | 15.7** |
| Panel H | 18.2 | 16** |

**Significant changes from baseline values

TABLE 10
Change in Assessed Age (Global Score) with Oral and Topical Product
(Average Global Age Score)

| Group | Before treatment | After 60 days |
|---|---|---|
| Panel A | 5.4 | 5.4 |
| Panel B | 5.3 | 4.43** |
| Panel C | 5.3 | 4.88** |
| Panel D | 5.25 | 4.76** |
| Panel E | 5.25 | 4.78* |
| Panel F | 5.3 | 4.2** |
| Panel G | 5.25 | 4.7** |
| Panel H | 5.5 | 4.65** |

**Significant changes from baseline values

Study 2: Placebo Controlled 60 Day Nutritional Supplement Study: Energizing Supplement Combined with Valerian Supplement to Assess Cell Renewal Rates and Deeper Lines and Wrinkles

Three groups of fifteen subject each, each group having an average age of fifty years plus with ages ranging from 45 to 60 years, ingested the inventive skin energizing nutritional supplement(s), once a day for a 60 day period. Some subjects also ingested a relaxing supplement once a day. Some of the subjects also used the exfoliating toner of Table 7 which was also designed to enhance skin cell renewal rates. At the study start and after 60 days, subjects were evaluated for cell renewal rate, superficial lines and wrinkles, and apparent relative age, as determined by a global age assessment method.

Fifteen subjects were grouped into three panels. Subjects ingested or were treated with the following compositions as described in Table 11. In accordance with the inventive skin care products were applied once a day to certain subjects in a composition as described above, and oral cell renewal supplement were taken once a day in the morning while a relaxation product was taken in the evening. All oral supplements were soft gelatin capsules.

TABLE 11

| Panel (Average Age) | Nutritional Product | Skin Care Product |
|---|---|---|
| A (55.1) | Composition 1(AM) | none |
| B (53.5) | Composition 1(AM), | none |
| | Composition 2(PM) | |
| C (54) | Composition 1(AM), | Exfoliating toner |
| | Composition 2(PM) | |

Subjects were evaluated before and after 60 days of treatment. Soft gelatin capsules were formulated with the following compositions and taken once a day for 60 days.

Composition 1 was a formulation of 100 mg rhodiola, 200 mg maca, 50 mg holy basil and 5 mg adenosine. Composition 2 was a formulation of 100 mg valerian root (valerian officinalis) at a composition of >0.8% valeric acid, 75 mg zizyphus jujube with a composition >2.5% tripertene, 50 mg passion flower (maltol, chysin) >3% flavonoids, glycosides and 25 mg quassia amara bark having 0.1% quassinoids.

Composition 2 was formulated with the following blend of relaxing herbs disclosed in U.S. Pat. No. 5,869,540, the disclosure of which is incorporated herein by reference. This blend included 100 mg Valeriana officinalis 100 mg (Valeric acid >3%), 100 mg Passiflora incarnate, 50 mg Magnolia acuminate. The dry powder blend was formulated into a two piece card gelatin based capsule as per standard methods know to those skilled in the art of capsule manufacture.

Results of Study 4 are shown in Table 12, Table 13 and Table 14. As shown in Table 12 cell renewal rates are not increased, beyond that seen with the morning supplement, by the use of the relaxing blend (Data Panels A versus B). Adding a topical exfoliant to the ingestible products (Panel C versus B) did however increase somewhat the cell renewal rates observed. Table 13 however shows that use of the relaxing supplement and the energizing supplement resulted in a greater wrinkle reduction (Data Panels A versus B). It was observed that predominantly the deeper wrinkles were dramatically diminished. Table 14 demonstrates that combining the energizing supplement with the relaxing supplement results in a greater improvement in global age assessment (Data Panel A versus B). Combining a topical exfoliant with the two oral products, only results in a modest improvement in facial appearance (Data Panel B versus C).

The cooperative interaction between the cell renewal and the relaxing supplement is believed to work in the following manner. The relaxing supplement makes skin look better but not making better skin per se. The inventive effects are directed towards the neuro-muscular junction and simply unfolding wrinkles that have been held in place by muscular tension. Skin metabolism is still slower than youthful skin, and skin will be thinner and need to be revitalized. The inventive treatments according to the present disclosure results in a stimulation of overall general metabolism in the epidermis and likely the dermis. By increasing this metabolism, cells are producing more energy, they reproduce more quickly and the skin becomes thicker, smoother and firmer. These effects mainly improve the more shallow and superficial wrinkles. However, when combined with the relaxing invention, the deeper wrinkles that have been unfolded, will have firmer, revitalized skin replacing the older, slowly metabolizing skin, and this makes the deeper wrinkle effect more permanent.

TABLE 12
Change in Cell Renewal Rates with Oral and Topical Product
(days for Dansyl chloride stain to disappear)

| Group | Before treatment | After 60 days |
|---|---|---|
| Panel A | 20.2 | 16.6** |
| Panel B | 20.7 | 16.8**# |
| Panel C | 20.3 | 14.3** |

**Significant changes from baseline values

TABLE 13
Change in Superficial facial lines with Oral and Topical Product
(Total SFL Score)

| Group | Before treatment | After 60 days |
|---|---|---|
| Panel A | 20.5 | 18.3** |
| Panel B | 20.4 | 15.6** |
| Panel C | 20.7 | 14.4** |

**Significant changes from baseline values

TABLE 14
Change in Assessed Age (Global Score) with Oral and Topical Product
(Average Global Age Score)

| Group | Before treatment | After 60 days |
|---|---|---|
| Panel A | 5.17 | 4.75** |
| Panel B | 5.25 | 4.23** |
| Panel C | 5.23 | 4.17** |

**Significant changes from baseline values

What is claimed is:

1. A dietary supplement composition comprising:
a therapeutically effective amount of at least one sirt 1 activating agent;
a therapeutically effective amount of at least one Qi activating agent; and
a therapeutically effective amount of at least one adaptogen.

2. The composition of claim 1, wherein said at least one sirt 1 activating agent is selected from the group consisting of resveratrol, extracts containing resveratrol such as grape skin, rhodiola rosea, Japanese Knotwood, wheat grass extract fisetin and 4-isoleucine.

3. The composition of claim 1, wherein said at least at least one Qi activating agent is selected from the group consisting of schisandra berry, glycerrhiza uralenis or glabra, red peony, astragali root, angelica root and maca.

4. The composition of claim 1, wherein said at least one adaptogen is selected from the group consisting of holy basil, lepedium meyenil and withania somnifera.

5. The composition of claim 1 further comprising an effective amount of at least one microcirculation increasing agent.

6. The composition of claim 5, wherein said at least one microcirculation increasing agent is selected from the group consisting of adenosine, cordyceps sinensis, nicotine and caffeine.

7. The composition of claim 1 further comprising a photoprotective agent.

8. The composition of claim 7, wherein said photoprotective agent is lycopene.

9. The composition of claim 1, wherein said therapeutically effective amount of at least one sirt 1 activating agent is about 100 mg of rhodiola, said therapeutically effective amount of at least one Qi activating agent is about 100 mg maca and said therapeutically effective amount of at least one adaptogen is about 25 mg holy basil.

10. The composition of claim 9 further comprising about 5 mg of adenosine.

11. The composition of claim 1, wherein said therapeutically effective amount of at least one sirt 1 activating agent is about 100 mg of rhodiola, said therapeutically effective amount of at least one Qi activating agent is about 50 mg schisandra berry and said therapeutically effective amount of at least one adaptogen is about 50 mg lepedium.

12. The composition of claim 11 further comprising about 50 mg cordyceps and about 5 mg caffeine.

13. The composition of claim 1, wherein said therapeutically effective amount of at least one sirt 1 activating agent is about 50 mg of resveratrol, said therapeutically effective amount of at least one Qi activating agent is about 50 mg wheat grass and about 50 mg red peony and about 50 mg angelica and said therapeutically effective amount of at least one adaptogen is about 50 mg withania.

14. The composition of claim 13 further comprising about 5 mg of adenosine.

15. The composition of claim 1, wherein said therapeutically effective amount of at least one sirt 1 activating agent is about 100 mg of resveratrol, said therapeutically effective amount of at least one Qi activating agent is about 100 mg schisandra berry and said therapeutically effective amount of at least one adaptogen is about 50 mg withania.

16. The composition of claim 1, wherein said therapeutically effective amount of at least one sirt 1 activating agent is about 25 mg of resveratrol, said therapeutically effective amount of at least one Qi activating agent is about 25 mg maca, about 25 mg red peony and about 25 mg wheat grass and said therapeutically effective amount of at least one adaptogen is about 25 mg withania.

17. The composition of claim 16 further comprising about 25 mg cordyceps.

18. A method for reducing the appearance of facial wrinkles comprising:
administering an oral composition comprising
a therapeutically effective amount of at least one sirt 1 activating agent;
a therapeutically effective amount of at least one Qi activating agent; and
a therapeutically effective amount of at least one adaptogen.

19. The method of claim 18, wherein said at least one sirt 1 activating agent is selected from resveratrol, extracts containing resveratrol such as grape skin, rhodiola rosea and wheat grass extract, fisetin and 4-isoleucine.

20. The method of claim 18, wherein said at least at least one Qi activating agent is selected from schisandra berry, glycerrhiza uralenis or glabra, red peony, astragali root, angelica root and maca.

21. The method of claim 18, wherein said at least at least one adaptogen is selected from holy basil, lepedium meyenil and withania somnifera.

22. The method of claim 18, wherein said oral composition further comprises an effective amount of at least one microcirculation increasing agent.

23. The method of claim 22, wherein said at least one microcirculation increasing agent is selected from adenosine, cordyceps sinensis, nicotine and caffeine.

24. The method of claim 18, wherein said oral composition further comprises a photoprotective agent.

25. The method of claim 18, wherein said photprotective agent is lycopene.

26. The method of claim 18, wherein said therapeutically effective amount of at least one sirt 1 activating agent is about 100 mg of rhodiola, said therapeutically effective amount of at least one Qi activating agent is about 100 mg maca and said therapeutically effective amount of at least one adaptogen is about 25 mg holy basil.

27. The method of claim 26, wherein said oral composition further comprises about 5 mg of adenosine.

28. The method of claim 18, wherein said therapeutically effective amount of at least one sirt 1 activating agent is about 100 mg of rhodiola, said therapeutically effective amount of at least one Qi activating agent is about 50 mg schisandra berry and said therapeutically effective amount of at least one adaptogen is about 50 mg lepedium.

29. The method of claim 28, wherein said oral composition further comprises about 50 mg cordyceps and about 5 mg caffeine.

30. The method of claim 18, wherein said therapeutically effective amount of at least one sirt 1 activating agent is about 50 mg of resveratrol, said therapeutically effective amount of at least one Qi activating agent is about 50 mg wheat grass and about 50 mg red peony and about 50 mg angelica and said therapeutically effective amount of at least one adaptogen is about 50 mg withania.

31. The method of claim 30, wherein said oral composition further comprises about 5 mg of adenosine.

32. The method of claim 31, wherein said therapeutically effective amount of at least one sirt 1 activating agent is about 100 mg of resveratrol, said therapeutically effective amount of at least one Qi activating agent is about 100 mg schisandra berry and said therapeutically effective amount of at least one adaptogen is about 50 mg withania.

33. The method of claim 18, wherein said therapeutically effective amount of at least one sirt 1 activating agent is about 25 mg of resveratrol, said therapeutically effective amount of at least one Qi activating agent is about 25 mg maca, about 25 mg red peony and about 25 mg wheat grass and said therapeutically effective amount of at least one adaptogen is about 25 mg withania.

34. The method of claim 33 further comprising about 25 mg cordyceps.

35. The method of claim 18, wherein administering comprises administering an oral dosage, twice a day for three months.

36. The method of claim 18, wherein administering comprises administering an oral dosage, once a day for 60 days.

37. The method of claim 18, further orally comprising a photoprotective mix including lycopene.

38. The method of claim 18, further comprising applying a topical formulation having exfoliants selected from the group consisting of actizyme, an acid protease lactic acid, glycolic acid and salicylic acid.

39. The method of claim 18, further comprising administering 25 mg valerian extract.

40. The method of claim 18, further comprising applying an herbal toner.

41. The method of claim 18, further comprising applying a topical cellulite product.

42. The composition of claim 1, further comprising herbal treatment for stress-related deep wrinkles comprising chronic oral administration for at least two weeks of a beverage containing an effective amount of a valerian root extract sufficient to provide a desired reduction in the appearance of said deep wrinkles.

43. The composition of claim 7, further comprising herbal treatment for stress-related deep wrinkles comprising chronic oral administration for at least two weeks of a beverage containing an effective amount of a valerian root extract sufficient to provide a desired reduction in the appearance of said deep wrinkles.

44. The composition of claim 8, further comprising herbal treatment for stress-related deep wrinkles comprising chronic oral administration for at least two weeks of a beverage containing an effective amount of a valerian root extract sufficient to provide a desired reduction in the appearance of said deep wrinkles.

45. The composition of claim 8 further comprising a photoprotective agent.

46. The composition of claim 44, further comprising herbal treatment for stress-related deep wrinkles comprising chronic oral administration for at least two weeks of a beverage containing an effective amount of a valerian root extract sufficient to provide a desired reduction in the appearance of said deep wrinkles.

47. The composition of claim 46, further comprising herbal treatment for stress-related deep wrinkles comprising chronic oral administration for at least two weeks of a beverage containing an effective amount of a valerian root extract sufficient to provide a desired reduction in the appearance of said deep wrinkles.

48. The method of claim 47, further comprising applying an herbal toner.
